(19) United States
(12) Patent Application Publication
(10) Pub. No.: US 2011/0059545 A1
(43) Pub. Date: Mar. 10, 2011

(54) TIME-TEMPERATURE INDICATOR BASED ON OLIGOMERIC SPIROAROMATICS

(75) Inventors: Husein Salman, Rajar (IL); Elena Tenetov, Nesher (IL); Leonhard Feiler, Binzen (DE); Thomas Raimann, Sisseln (CH)

(73) Assignee: FRESHPOINT HOLDINGS SA, La Chaux-de-Fonds (CH)

(21) Appl. No.: 12/523,110

(22) PCT Filed: Jan. 14, 2008
(86) PCT No.: PCT/EP08/50323

§ 371 Date: Nov. 30, 2010

(30) Foreign Application Priority Data
Jan. 22, 2007 (EP) 07100873.4

(51) Int. Cl. G01N 21/00; C07D 209/96

(52) U.S. Cl. 436/164; 548/409

(57) ABSTRACT

The present invention relates to a time temperature indicator for indicating a temperature change over time comprising one dimeric or trimeric spiropyran indicator of the formula I or II wherein R₁-R₄ independently of one another is hydrogen, —C₁C₆ alkoxy, halogen, CF₃, —C₁C₆ alkyl or —NO₂, R₅ is hydrogen, halogen, —C₁-C₆ alkoxy, —COOH, —COO—C₁-C₆ alkyl, —CF₃ or phenyl; R₁₁ is hydrogen or R₁₁ and R₅ form together a phenyl ring; Rₐ is —C₁-C₆ alkyl R_{b} is —C₁-C₆ alkyl, or together with Rₐ form a 5-6 membered ring L is a divalent linker; L′ is a trivalent linker.

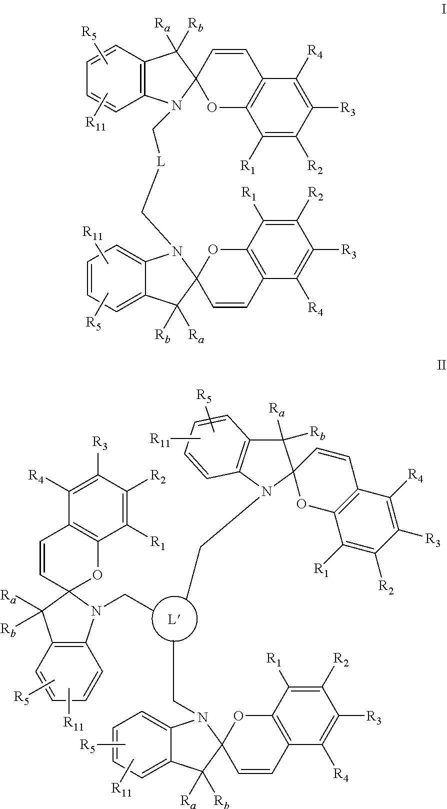

### FIELD OF THE INVENTION

The present invention relates to a time temperature indicator (TTI) for indicating the elapsed time-temperature comprising at least one oligomeric spiroaromatic compound. More particularly the invention provides photochromic oligomeric spiropyran compounds as well as methods for their preparation and use as active ingredients of TTI.

### BACKGROUND OF THE INVENTION

Time-temperature indicators, TTIs, are substrates for packaging of or attachment to perishable goods that are capable of reporting the sum of the partial or full time temperature history of any good to which it is thermally coupled.

Temperature abuse is one of the most frequently observed causes for predated goods spoilage. It is therefore important and desired to monitor the time-temperature history of such perishable goods, preferably, using inexpensive and consumer friendly means. Time temperature indicators are substances that are capable of visually reporting on the summary of the time temperature history of the substance, and consequently, of the perishable good it is associated with. Designed mainly for the end user, time temperature indicators are usually designed to report a clear and visual Yes/No signal.

Some examples of photochromic bis-spiropyran compounds are presented in the literature. E. Gonzalez et al, J. Appl. Polymer Science, 71 (199) 259-266 describe microwave assisted preparation of bis-spiropyrans and the photochromic effect of polyurethane-acrylate block copolymers containing 6-nitro-bis-spiropyranes, for example 6-nitro bis p-xylene spiropyran or 6-nitro bis decyl spiropyran.

In another work (Young Jin Cho et al in Dyes and pigments 44 (2000 19-25) is described synthesis of bis-spirocompounds in which two spiropyrans are linked by an ethynyl group. U.S. Pat. No. 6,747,145 discloses photochromic bis-naphthopyrans linked to oligo-thiophenes. Bis-spirooxazines contained different phenylene linkers are described in EP 0321563.

WO 99/39197 describes the use of photochromic dyes, based on a transfer reaction as active materials for TTIs. TTIs based on these materials are highly accurate and reproducible and can be charged using stimulating light. It further teaches that by placing a special filter atop the active substance most of the UV and visible spectrum of light can be filtered which prevents undesired re-charging and photobleaching of the TTI.

WO 2005/075978 teaches TTIs based on photochromic indicator compounds. The photo-chromic reactions of the TTIs taught in WO 2005/075978 are valence isomerization reactions without migration of an atom or chemical group attached to the indicator compound in a time and temperature dependent manner. Preferred indicator compounds include diarylethenes and spiroaromatics. The spiroaromatic compounds used in WO 2005/075978 are monomers.

There is a need for a commercial TTI based on indicator compounds that have an improved pigmentation ability and longer lifetime than its monomeric analogs. The information drawn from the TTI must be highly accurate and reproducible, particularly said information must be proportional to the time-temperature history. Finally, such a TTI should be printable on commercially used substrates, for example packaging materials for food items and further, the TTI should be stable enough to allow storage at room temperature before its activation.

It has now been found that a time-temperature indicator (TTI) system that is based on dimeric or trimeric spiroaromatic compounds shows improved lifetime.

### DETAILED DESCRIPTION OF THE INVENTION AND PREFERRED EMBODIMENTS

The invention relates to a time temperature indicator for indicating a temperature change over time comprising at least one dimeric or trimeric spiropyran indicator of the formula I or II

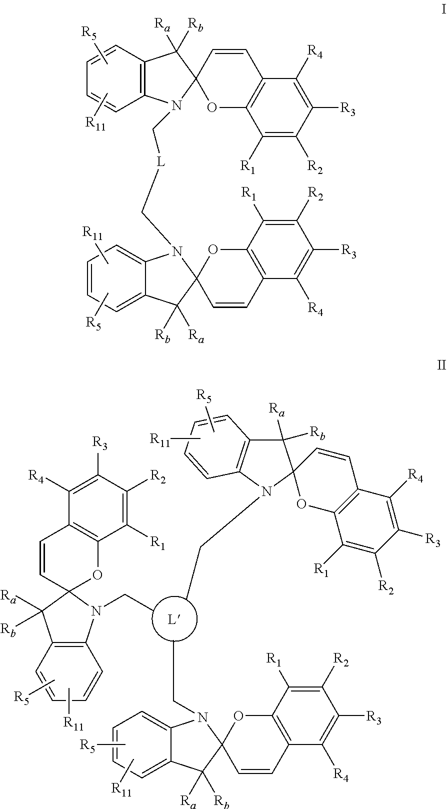

wherein
R₁-R₄ independently of one another is hydrogen, —C₁-C₆ alkoxy, halogen, CF₃, —C₁-C₆ alkyl or —NO₂
R₅ is hydrogen, halogen, —C₁-C₆ alkoxy, —COOH, —COO—C₁-C₆alkyl, —CF₃ or phenyl;
R₁₁ is hydrogen or R₁₁ and R₅ form together a phenyl ring;
Rₐ is —C₁-C₆ alkyl
R_{b} is —C₁-C₆ alkyl, or together with Rₐ form a 5-6 membered ring
L is a divalent linker;
L′ is a trivalent linker.

In one embodiment the spiroaromatic compound is trimeric. (Claim 2)

A spiropyran trimer of the formula II is for example

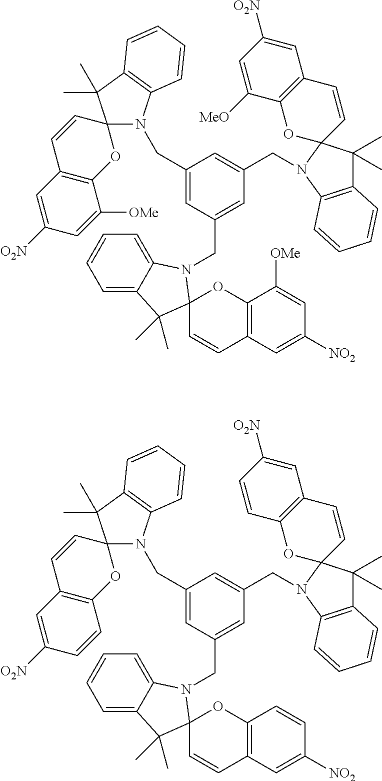

Preferred are compounds of the formula I. (Claim 3)

In a preferred embodiment the present invention provides a time temperature indicator comprising at least one dimeric spiroaromatic compound of the formula I wherein

R₁ is hydrogen, —C₁-C₆ alkoxy, halogen, —C₁-C₆ alkyl or —NO₂,
R₂ is hydrogen or —C₁-C₆ alkoxy;
R₃ is NO₂ or halogen;
R₄ is hydrogen, —C₁-C₆ alkoxy or halogen;
R₅ is hydrogen, halogen, methoxy or —COOH
R₁₁ is hydrogen,
Rₐ is methyl or ethyl,
R_{b} is methyl or ethyl,
L is a divalent linker.

The term “divalent linker” or “trivalent linker” as used herein refers to any divalent or trivalent group capable of linking two or three spiropyran moieties together.

Examples of divalent linker groups are selected from C₁-C₁₂ alkylene, C₁-C₁₂ alkenylene, C₁-C₁₂ alkynylene,

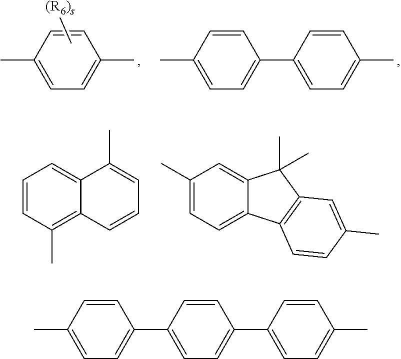

wherein R₆ is hydrogen, halogen, —C₁-C₆ alkoxy, CF₃, NO₂, preferably methoxy or hydrogen.
s. is 1-4, preferably 1 or 2

Examples of trivalent linker groups are

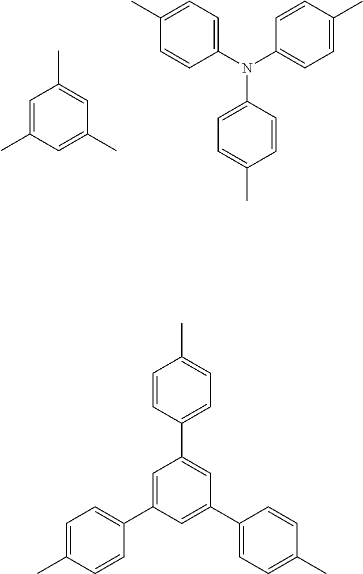

C₁-C₆ alkoxy is preferably methoxy. The term “halogen” refers to fluoro, chloro, bromo or iodo.

More preferred:

R₁ is hydrogen or methoxy.
R₂ is hydrogen or methoxy.
R₃ is nitro.
R₄ is hydrogen.
R₅ is hydrogen, halogen, methoxy or —COOH,
Rₐ is methyl.
R_{b} is methyl.

The examples of bis-spiropyran compounds of the formula I wherein R₃ is NO₂, R₄ is H, are presented in Table 1

TABLE 1

| Ex. | L | R5 | R1 | R2 |
|---|---|---|---|---|
| 127 | 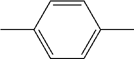 | H | MeO | H |
| 129 | 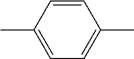 | Br | MeO | H |
| 262 | 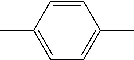 | MeO | MeO | H |
| 343 | 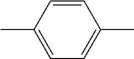 | H | MeO | MeO |
| 130 | 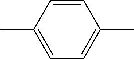 | H | H | H |
| 131 | 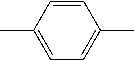 | Br | H | H |
| 357 | 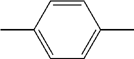 | COOH | MeO | H |
| LF3427 | 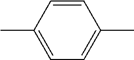 | forms together with R₁₁ a phenyl ring | MeO | H |
| 140 | 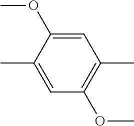 | H | MeO | H |
| 143 | 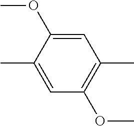 | H | H | H |
| 173 | 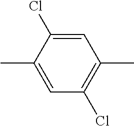 | H | MeO | H |
| 174 | 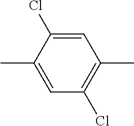 | H | H | H |
| 194 | 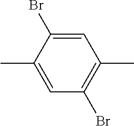 | H | MeO | H |
| 335 | 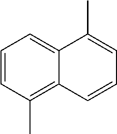 | H | MeO | H |
| 156 | 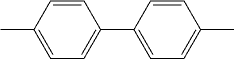 | H | MeO | H |
| 157 | 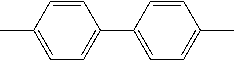 | H | H | H |
| 360 | 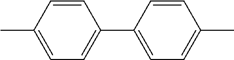 | COOH | MeO | H |
| 183 | 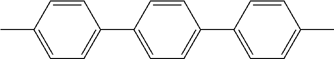 | H | MeO | H |
| 184 | 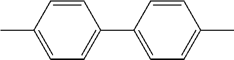 | H | H | H |
| LF3375 | —CH₂—CH═CH—CH₂— | H | MeO | H |
| LF 3376 | —CH₂—CH₂—CH₂— | H | MeO | H |
| 124 | —CH₂—CH₂—CH₂—CH₂— | H | MeO | H |

Best results have been obtained with the following bis-spiropyrans:

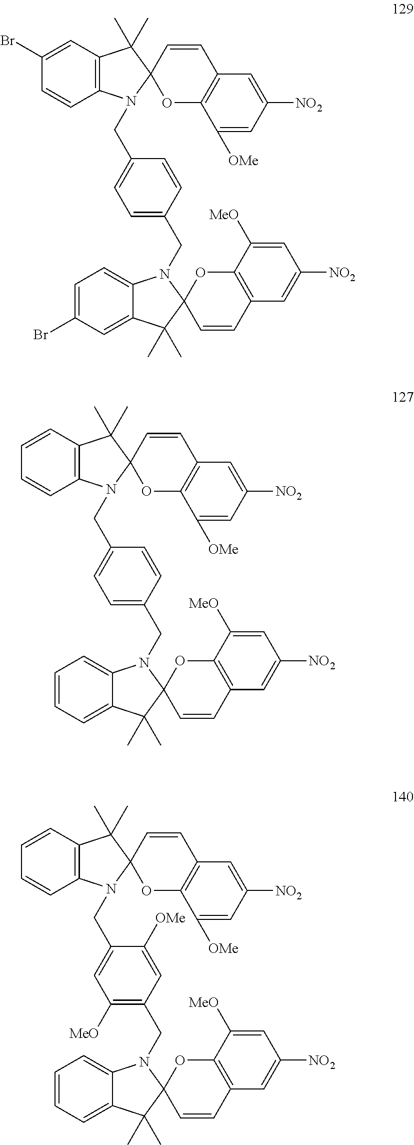

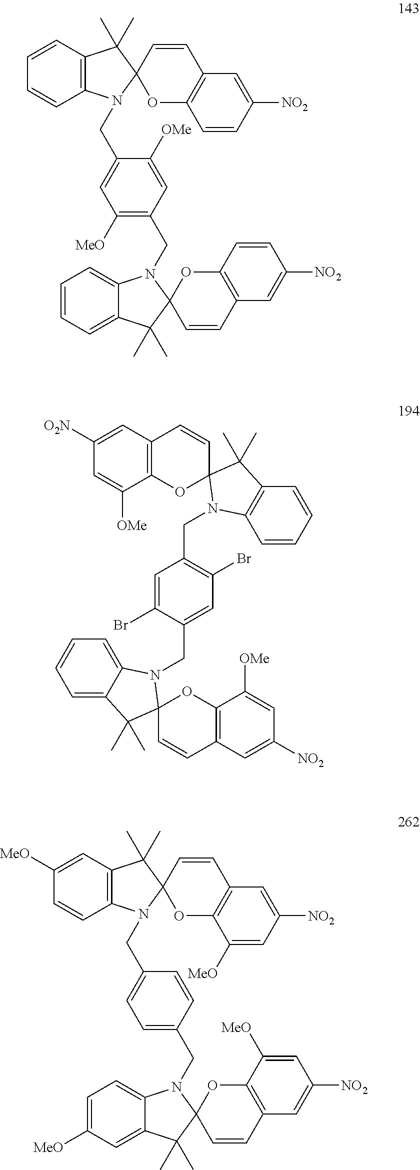

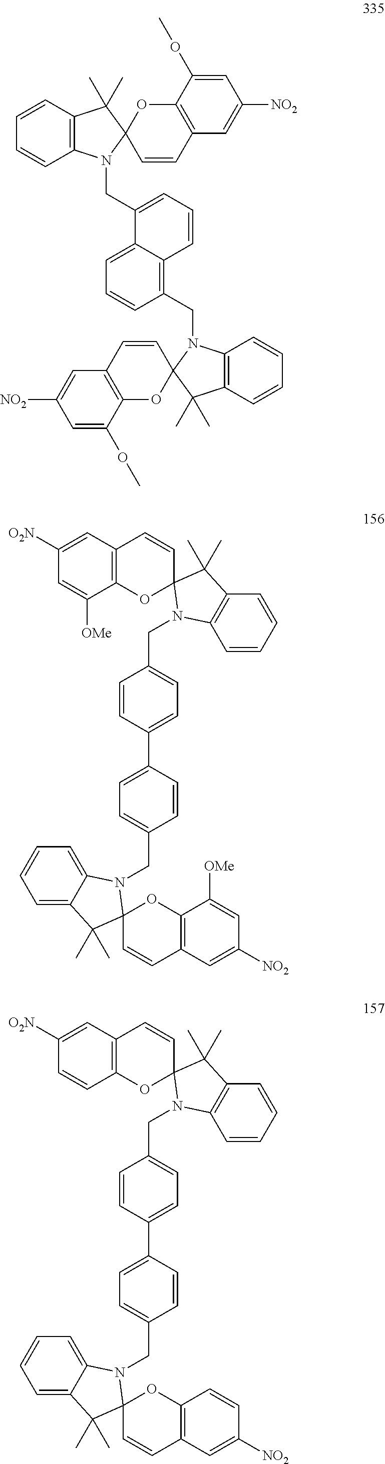

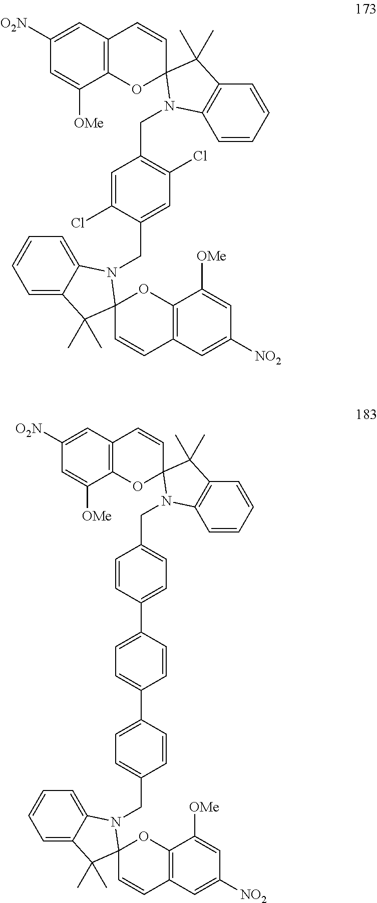

Claim 4.

Especially preferred is compound 127, which preparation is disclosed in Example 2.

Further interesting compounds are:

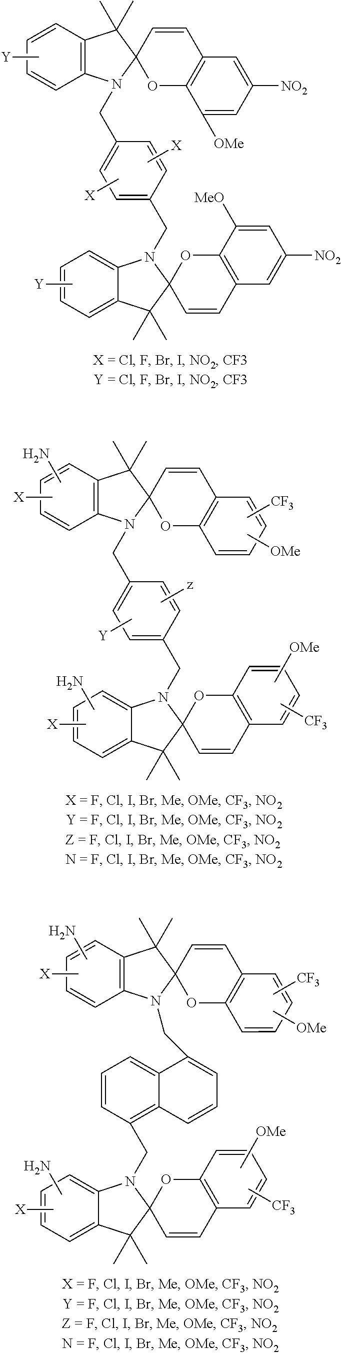

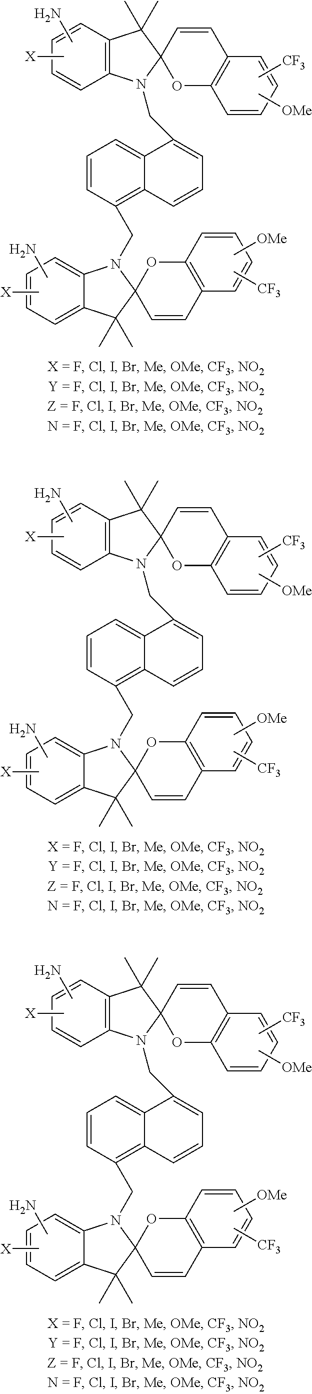

The indicator compounds of formula I or II are reversibly photochromic (Scheme 1).

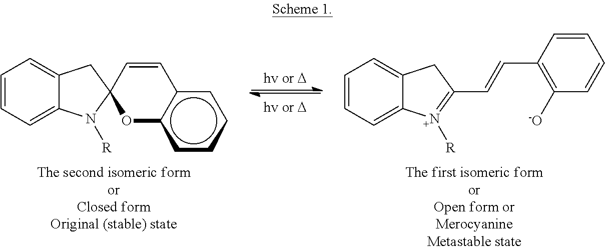

By virtue of its photochromic properties, the indicator compound can undergo photo-induced coloration by irradiation with photons of a specific energy range (conversion of the second isomeric form, thermodynamically more stable) into the first isomeric form (open form) the coloration being followed by a time- and temperature-dependent decoloration (conversion of the first isomeric form into the second isomeric form).

The coloration of the indicator compound can take place at a defined time point, preferably, for example, immediately after printing onto a substrate, such as the packaging of a perishable material.

In oligomeric spiropyrans there are at least two different metastable isomers. At least two distinct valence isomeric forms exist in each spiroaromatic unit of the oligomeric indicator. These isomeric forms are at least one colored open form, first isomeric form, and at least one colorless cyclic form (closed form or second isomeric form).

Suitable active materials exhibit the following characteristics:
(1) the system has at least one thermal process leading from one metastable state to one stable state, where the two states of the spiroaromatic compounds are characterized by a distinctly different color and/or any other measurable physical parameter such as luminescence, refraction index, conductivity and the like.
(2) the stable state may be converted into the metastable state using one or any combination of stimuli, among others the following processes:
   a) photonic induction,
   b) thermal induction,
   c) pressure induction,
   d) electrical induction, or
   e) chemical induction; and
(3) other than temperature and photoinduction (in the visible light range), the metastable state is substantially not affected by anyone or any combination of stimuli such as a) photo induction, b) piezo induction, c) electro induction, d) chemo induction.

Photoinduction means that the initially colourless indicator is irradiated with light, preferably in the UV or near-UV range, as a result a reversible internal valence isomerisation from a colourless inactivated state to a coloured activated one is induced. A reverse discolouration process then proceeds at a rate that is time and temperature dependent.

The metastable state may further be achieved by pressure induction. In this procedure, the matrix embedded with and/or atop the substance is passed between two bodies, such as metal rolls, which apply pressure onto the surface of the matrix thereby inducing the formation of the metastable state. By adjusting the time and pressure imparted by the bodies to the active material, it is possible to control the degree of conversion from a stable state to a metastable state in the TTI active matrix.

The metastable state may be achieved by thermal induction. In this particular induction process, the matrix embedded with the substance to be induced is heated to temperatures normally below the melting point of said substance. The heat may be applied by any method known such as, but not limited to, a thermal transfer printing head. In one specific case, the heat is applied to the matrix while being passed through two heated metal rolls. In this case, the pressure applied to the surface is not capable itself of inducing the formation of the metastable state, but serves merely to ensure controlled thermal contact between the heaters and the sample. The metastable state is achieved as a result of the heat transfer from the heaters, i.e., the metal rolls, which are in contact with the matrix and the matrix itself.

However, there may be instances where the use of any combination of pressure, light and thermal inductions may be desired or necessary. It is therefore, a further embodiment of the present invention, to achieve the metastable state of the substances to be used with the TTIs of the present invention, by a combination of stimuli.

The active material of the present invention may be in the form of a crystal or a poly-crystalline powder, in which the forward and reverse reactions take place or alternatively may be in a form of any other condensed phase such as a glass, a polymer solution or attached to a polymer, or in the form of a liquid or a solution.

In yet another aspect of the present invention, there is provided a method for the manufacture of a TTI comprising at least one of the spiroaromatic indicator compounds of the formula I or II in form of a pigment or a dye; said method comprising the steps of
(a) introducing into a matrix or atop a matrix a dimeric or trimeric spiropyran indicator of the formula I or II as defined in claim 1 and
(b) converting the spiropyran indicator from an original stable state into a metastable state by a process selected from photonic induction, thermal induction, pressure induction, electrical induction, or chemical induction,
(c) optionally applying a protector film.
(Claim 6)

The converting step b may be effected immediately after step a) or later at any time.

The original stable state and the metastable state is defined above (Scheme 1 above)

The term “introducing into a matrix” means any form of admixing the TTI indicator into a matrix, for example, indicator-doping of the matrix, sol-gel embedment of the indicator in the matrix, embedment of the indicator as small crystallites, solid solution and the like.

The matrix used in the present invention may be a polymer, an adhesive, all kinds of paper or cardboard, all kinds of printing media, metal, or any glass-like film.

The matrix is also called substrate.

Examples of printing media may be self-adhesive PP, cold lamination films, PVC films, PPpaper, glossy photo paper, vinyl sheets and the like; inkjet media.

The matrix polymer is a high molecular weight organic material may be of natural or synthetic origin and generally has a molecular weight in the range of from 10³ to 10⁸ g/mol. It may be, for example, a natural resin or a drying oil, rubber or casein, or a modified natural material, such as chlorinated rubber, an oil-modified alkyd resin, viscose, a cellulose ether or ester, such as cellulose acetate, cellulose propionate, cellulose acetobutyrate or nitrocellulose, but especially a totally synthetic organic polymer (thermosetting plastics and thermoplastics), as are obtained by polymerisation, polycondensation or polyaddition, for example polyolefins, such as polyethylene, polypropylene or polyisobutylene, substituted polyolefins, such as polymerisation products of vinyl chloride, vinyl acetate, styrene, acrylonitrile, acrylic acid esters and/or methacrylic acid esters or butadiene, and copolymerisation products of the mentioned monomers, especially ABS or EVA. From the group of the polyaddition resins and polycondensation resins there may be mentioned the condensation products of formaldehyde with phenols, so-called phenoplasts, and the condensation products of formaldehyde with urea, thiourea and melamine, so-called aminoplasts, the polyesters used as surface-coating resins, either saturated, such as alkyd resins, or unsaturated, such as maleic resins, also linear polyesters and polyamides or silicones. The mentioned high molecular weight compounds may be present individually or in mixtures, in the form of plastic compositions or melts. They may also be present in the form of their monomers or in the polymerised state in dissolved form as film-forming agents or binders for surface-coatings or printing inks, such as boiled linseed oil, nitrocellulose, alkyd resins, melamine resins, urea-formaldehyde resins or acrylic resins.

The term “introducing” means also printing. In this case, the TTI is transformed into a printable ink.

The ink may directly be printed onto a matrix or directly onto the packaging material or label.

Thus, the present invention further concerns a printing ink or printing ink concentrate, comprising at least one spiropyran indicator of the formula (I) or (II) as defined in claim 1; for manufacturing a time temperature indicator. (claim 9)

Any of the printing methods known in the art can be used, e.g., ink jet printing, flexo printing, laser printing, thermo-transfer printing, pad printing, printing using cold lamination techniques, and the like.

In another embodiment, the indicator compound is part of a thermal transfer (TTR) ink composition and is transferred to the printed surface by applying heat to the TTR layer. By means of a reference scale printed with the time-temperature integrator, absolute determination of quality grades is possible. The time-temperature integrator and the reference scale are advantageously arranged on a light-colored substrate in order to facilitate reading.

It is possible to apply, preferably in black ink, further text (or information), such as an expiry date, product identification, weight, contents etc.

The reference color may be changed as one means for changing the lifetime of the TTI.

The time-temperature indicator may be covered with a protective film, designed to avoid photo recharging and/or photo bleaching.

Either the TTI or the filter may be printed using cold lamination techniques or pad printing techniques.

The protective film is, for example, a color filter, e.g. a yellow filter, which are permeable only to light having typical wavelengths that are longer than 430 nm.

Suitable filters are disclosed in the International application EP2007/060987, filed Oct. 16, 2007. Disclosed therein is a composition comprising at least one ultraviolet light and/or visible light absorbing layer which is adhered to an underlying layer containing a photo-chromic colorant,

which photo chromic colorant is activated by exposure to UV light to undergo a reversible color change, which color reversion occurs at a rate that is dependent on temperature,
wherein the light absorbing layer comprises a binder, from 1 to 60% by weight based on the total weight of the layer of an ultraviolet light absorber selected from the group consisting of hydroxyphenylbenzotriazole, benzophenone, benzoxazone, α-cyanoacrylate, oxanilide, tris-aryl-s-triazine, formamidine, cinnamate, malonate, benzilidene, salicylate and benzoate ultraviolet light absorbers.

If desired an irreversible photo-sensitive indicator can be applied as tamper-proofing in the form of a covering over the time-temperature integrator. Suitable irreversible indicators include, for example, pyrrole derivatives, such as 2-phenyl-di(2-pyrrole)methane. Such a material turns irreversibly red when it is exposed to UV light.

The invention further relates to a method of time temperature indication by converting the spiropyran indicator of the formula I or II as defined in claim 1 from an original stable state into a metastable state by a process selected from photonic induction, thermal induction, pressure induction, electrical induction, or chemical induction and detecting the time temperature dependent re-conversion from the metastable state to the original stable state. (claim 7)

The time temperature detection may be achieved optically by detecting a change in an optical property (such as for example absorption, transmission, reflectivity) of the TTI device. For instance, a color change is determined either visually by comparing to a reference sample, or using a colorimeter or any colour reading or colour comparing technique. (claim 8)

Preparation of Oligomeric Spirocompounds

The photochromic spiropyran compounds of the present invention may be prepared according to synthetic routes known in the literature.

The syntheses of bis-spirocompounds represented by formula I involve the process illustrated in Reactions A through E shown below and start from 2,3,3-trimethylindolenines which are commercially available (R₅═H) or readily prepared by Fisher's reaction.

Reaction A.

### Preparation of 5-substituted 2,3,3-trimethylindolenines of Formula III by Fisher's Reaction

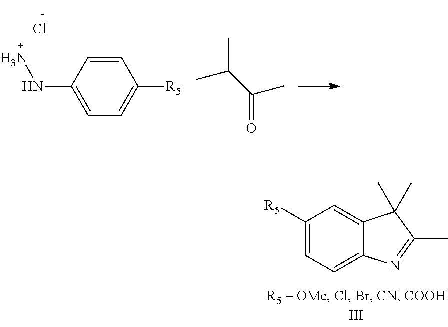

The reaction conditions are the standard ones described in the literature (Berman, E., Fox, R. E. and Thomson, F. D. Photochromic spiropyrans. I. The effect of substituents on the rate of ring closure. J. Am. Chem. Soc. 81, 1959, 5605-5608).

Reaction B

In Reaction B homobifunctional aromatic compounds may be prepared either by bromomethylation (Method I) or by radical bromination (Method II) of corresponding aromatic compounds.

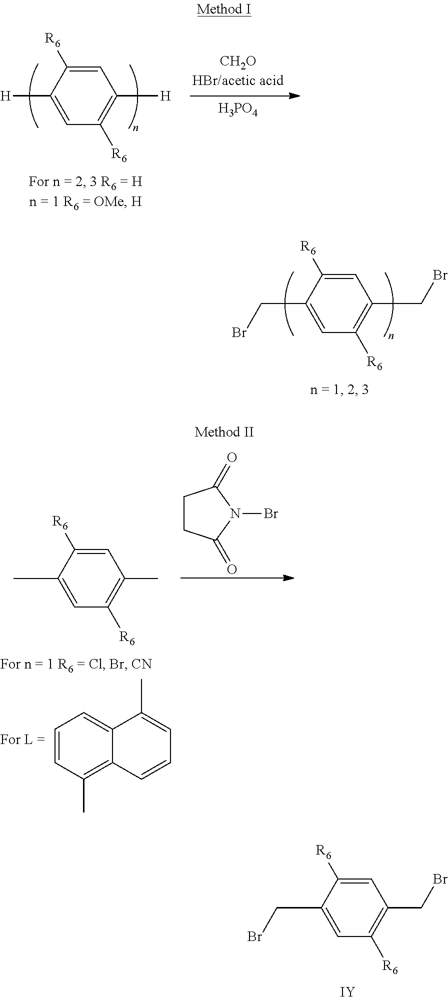

According to Method I an aromatic compound reacts with paraformaldehyde and hydrogen bromide in acetic acid in the presence of orthophosphoric acid under heating to give bifunctional compound represented by formula IY. The reaction conditions of the process are described in J. Am. Chem. Soc. 1992, 114: 6227-6238.

Alternatively, compounds of formula IY may be prepared according to Method II using N-bromosuccinimide (NBS) in suitable non polar solvent, preferably benzene, chloroform, carbon tetrachloride, chlorobenzene, more preferably, benzene and chlorobenzene.

Reaction C:

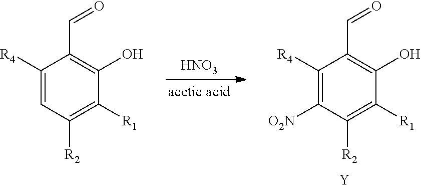

In Reaction C substituted salicylaldehyde represented by formula Y (the substituents R₁, R₂, and R₄ are the same as defined hereinabove) is dissolved in mixture of acetic acid and suitable organic solvent (such as dichloromethane, chloroform or the like) in ratio 1:1. The solution is treated with mixture of acetic and nitric acids under cooling with ice-water bath, to give after aqueous work up 5-nitrosubstituted salicylic aldehyde. Nitric acid concentration used in the process may be 100% or 70%, preferably 100%.

Reaction D

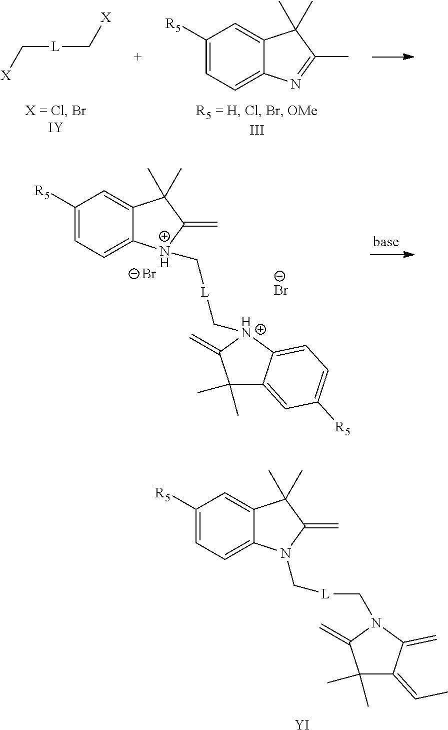

In reaction D indolenine of formula III reacts with bis-halomethyl compound represented by formula IY in an appropriate organic solvent (benzene, toluene, methylethylketone, acetonitrile, dioxane or a combination thereof) to give Fisher' base in the form of dihydrohalogenide. The reaction temperatures may be 80-120° C., preferably 85-90° C., reaction time may be about 10 h to about 3 days. The dihydrohalogenide of the Fisher' base YI is dissolved in dichloromethane and treated by aqueous solution of inorganic base (sodium hydroxide, sodium or potassium carbonate), to afford the free base YI, which is subjected to the next step without delay (because of the easiness of oxidation). Alternatively, the reaction may be carried out in the presence of organic (such as diisopropylethylamine, or other sterically hindered amines) or inorganic bases (such as potassium or sodium carbonates) to generate free base YI directly in the reaction mixture.

Reaction E.

In reaction E bis-spyropyran compounds may be formed from free Fisher's bases and the corresponding substituted salicylic aldehydes under reflux in suitable organic solvents (ethanol, acetonitrile, methylethylketone or dioxane)

The preferred embodiments of the present invention are illustrated by the following examples, which are in no way intended to limit the scope of the present invention.

### EXAMPLES

### Example 1

### Compound 156

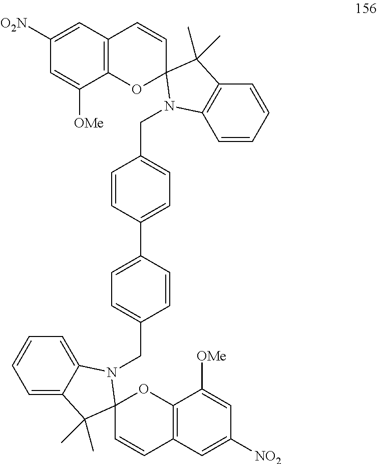

Step 1

Reaction B:

Biphenyl (15.4 g, 100 mmol) and paraformaldehyde (7.5 g, 250 mmol) were transferred into a 250 ml round bottom flask. HBr (33% in acetic acid, 100 ml, 579 mmol) and H₃PO₄ (20 ml) were added dropwise. The reaction mixture was stirred vigorously for 15 h at 80° C. under nitrogen. An additional aliquot of paraformaldehyde (2.5 g, 80 mmol) was added and the temperature raised to 120 C for 2 h. The reaction mixture was cooled to room temperature, the solids were filtered, washed with hexanes, recrystallized from benzene/hexane to afford 4,4′-Bis(bromomethyl)-1,1′biphenyl. Yield 5.4 g (15.9%)

Step 2

Step 2 involves the process described hereinabove as Reaction D:

A solution of 4,4′-Bis(bromomethyl)-1,1′biphenyl (2.50 g, 7.4 mmol) and 2,3,3-trimethylindolenine (2.58 g, 16.1 mmol, 2.60 ml) in toluene (30 ml, AR) was stirred for 48 h at 80-85° C. An additional portion of the indolenine (1 g, 0.85 eq) was added and the reaction mixture was stirred for an additional 48 h. The reaction mixture was cooled to room temperature. A solid was filtered, washed with ether, THF, ether, affording 5.0 g of the crude 4,4′-bis((3,3-dimethyl-2-methyleneindolin-1-yl)methyl)-1,1′biphenyl as dihydrobromide.

Step 3

Step 3 involves the process described hereinabove as Reaction E.

A solution of the 4,4′-bis((3,3-dimethyl-2-methyleneindolin-1-yl)methyl)-1,1′biphenyl dihydrobromide, (0.80 g, 1.6 mmol) in dichloromethane was treated with 5% NaOH under stirring for 0.5 h. The organic phase was separated, dried over Na₂SO₄, chromatographed on an alumina column in Hexane-CH₂Cl₂ (10-35%). Fractions containing free base were collected and the solvent was evaporated under reduced pressure (bath temp. 30° C., cooling under nitrogen). The self-crystallized free base was immediately suspended under heating in 50 ml ethanol containing a few drops of Et₃N.

3-methoxy-5-nitrosalicylaldehyde (0.65 g, 3.3 mmol) was added to the free-base solution under heating and stirring. The reaction mixture was refluxed for 1 h, cooled to room temperature, and filtered through a glass filter. The solid product was washed with ethanol, triturated with Et₃N (aq, 1%), washed with ethanol and hexane, and finally dried under vacuum to give bis-spiropyran 156. Yield 58%. The structure was confirmed by NMR and MS analysis.

### Example 2

### Compound 127

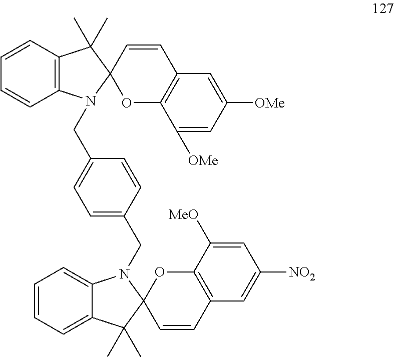

Step 1

The process of Step 2 in example 1 was followed except that α,α′-dibromoxylene was used instead of 4,4′-bis(bromomethyl)-1,1′-biphenyl. The reaction mixture was stirred for 60 h. 1,4-bis((3,3-dimethyl-2-methyleneindolin-1-yl)methyl)-benzene as dihydrobromide was obtained with 69% yield.

Step 2

The process of Step 3 in example 1 was followed except that 1,4-bis((3,3-dimethyl-2-methyleneindolin-1-yl)methyl)benzene dihydrobromide was used instead of 4,4′-bis((3,3-dimethyl-2-methyleneindolin-1-yl)methyl)-1,1′-biphenyl dihydrobromide. Crude product was triturated with ethanol overnight, dried in vacuo to afford bis-spiropyran compound 127. Yield 67%.

The structure was confirmed by NMR and MS analysis.

### Example 3

### Compound 194

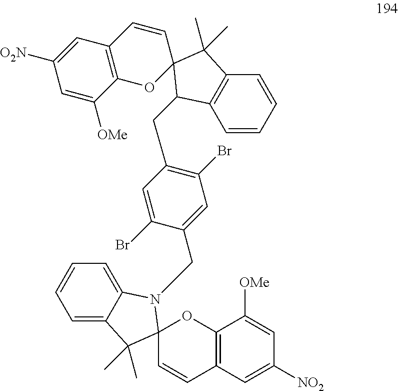

Step 1

Step 1 involves the process described hereinabove as Reaction B, Method II. 2,5-dibromo-p-xylene (10 g, 38 mmol) was dissolved in benzene (70 ml). Then NBS (14 g, 2.1 eq) and dibenzoyl peroxide (0.1 g, dried between two sheets of filter paper) were added and the mixture was refluxed under nitrogen. After 24 h, the succinimide was filtered off and the solvent was evaporated. The product was dissolved in chloroform, the solvent was partially evaporated and the crystals were formed under cooling. Crude product (6.7 g) was recrystallized from chloroform-hexane, giving rise to 5.0 g (31.2%) of pure 1,4-bis(dibromomethyl)-2,5-dibromobenzene. NMR spectrum conforms to the structure.

Step 2

The process of Step 2 in example 1 was followed with exception that 1,4-bis(dibromomethyl)-2,5-dibromobenzene was used instead of 4,4′-bis(bromomethyl)-1,1′biphenyl. The reaction mixture was filtered, washed with ether. Mother liquids and washings were joined, evaporated under reduced pressure, a residue was chromatographed on alumina (hexane-dichloromethane (0-30%) to give 1,4-bis((3,3-dimethyl-2-methyleneindolin-1-yl)methyl)-2,5-dibromo-benzene, which was subjected to the next step immediately.

Step 3

The process of Step 3 in example 1 was followed except that 1,4-bis((3,3-dimethyl-2-methyleneindolin-1-yl)methyl)-2,5-dibromo-benzene instead of 4,4′-bis((3,3-dimethyl-2-methyleneindolin-1-yl)methyl)-1,1′biphenyl. Yield 50%. NMR spectrum of the product conforms to the structure of bis-spirocompound FPSP194.

### Example 4

### Compound 335

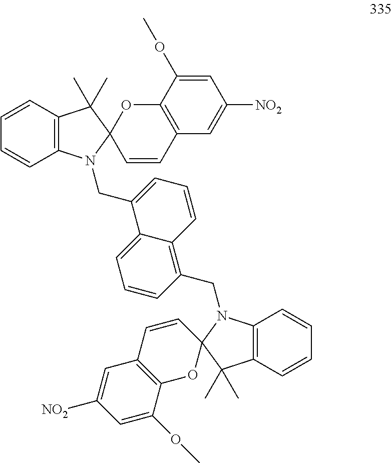

Step 1

The process of the Step 1 in example 3 was followed except that 1,5-dimethylnaphthalene (5.0 g, 32 mmol) was used instead of 2,5-dibromo-p-xylene. The reaction mixture was re-fluxed for 1 h (TLC monitoring: starting material disappeared after 0.5 h), cooled to room temperature; a precipitate was filtered, washed with benzene, suspended in 250 ml of water, washed with water for 45 min, filtered, dried giving rise to a crude product (˜10 g) which was crystallized from ethyl acetate to give 7.1 g (70.6%) of the pure bis-compound. NMR spectrum showed that the resulted product has the structure consistent with 1,5-dibromo-naphthalene.

Step 2

A mixture of 2,3,3-trimethylindolenine, 1,5-dibromomethylnaphthalene and potassium carbonate was heated at 90 C in 20 ml toluene for 48 h. Then the reaction mixture was filtered through alumina pad, alumina was washed with toluene. The joined filtrate and washings were evaporated under reduced pressure, a residue was chromatographed on alumina (Hexane-dichloromethane 0-10%). Fractions containing the product were collected and evaporated to give pure 1,5-bis((3,3-dimethyl-2-methyleneindolin-1-yl)methyl)-naphthalene which was subjected to the next step without delay.

Step 3

The process of Step 3 in example 1 was followed except that 1,5-bis((3,3-dimethyl-2-methyleneindolin-1-yl)methyl)-naphthalene was used instead of 4,4′-bis((3,3-dimethyl-2-methyleneindolin-1-yl)methyl)-1,1′biphenyl. The reaction mixture was refluxed overnight, cooled to room temperature, filtered, washed with ethanol, water, triturated with n-butanol, washed with ethanol, hexane, dried in vacuo, giving rise to light grey-greenish powder of FPSP335. Yield 81%. The NMR spectrum conforms to the structure.

### Example 5

### Compound 183

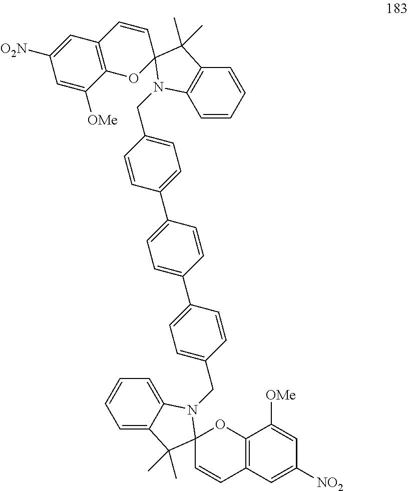

Step 1

The process of Step 1 in Example 1 was followed except that p-terphenyl was used instead of 1,1′-biphenyl. Molar ratio—terphenyl:paraformaldehyde:HBr—1:6:8. The reaction mixture was heated at 80 C for 16 h under nitrogen. Then the temperature was raised to 120 C for 8 h, the reaction mixture was cooled to room temperature, solids were filtered, washed with acetone dried on a glass filter, to give crude 4,4″-bis-bromomethyl-[1,1′;4′,1″]terphenyl. The crude was repeatedly extracted with boiling toluene. The hot toluene solution was filtered and the product was crystallized under cooling to room temperature, filtered, dried in vacuum, giving rise to of 4,4″-bis-bromomethyl-[1,1′;4′,1″]terphenyl (23% yield (compound 181).

Step 2

A mixture of 2,3,3-tri-methyl-indolenine (4.29 g, 4.2 ml, 26.9 mmol), 4,4″-bis-bromomethyl-[1,1′;4′,1″]terphenyl (3.2 g, 7.69 mmol) and potassium carbonate (3.72 g. 26.9 mmol) in 50 ml of dioxane was heated at 90 C for 48 h, cooled to room temperature. The solvent was evaporated; a residue was partitioned between dichloromethane and 5% NaOH (aq), organic layer was separated, water layer was back extracted with dichloromethane, joined organic phases were dried over Na₂SO₄, concentrated under reduced pressure, chromatographed on alumina. Fractions containing bis-product (R_{f}=0.7, Slilca, dichloromethane-hexane—1:1) were collected, evaporated to dryness, to give crude free base 182 (yellow solid) which was subjected to the next step immediately.

Step 3

The process of Step 3 in example 1 was followed except that 4,4″-bis((3,3-dimethyl-2-methyleneindolin-1-yl)methyl)-[1,1,4′,1″]terphenyl was used instead of 4,4′-bis((3,3-dimethyl-2-methyleneindolin-1-yl)methyl)-1,1′biphenyl. The reaction mixture was cooled to room temperature, filtered through glass filter; solid product was washed with ethanol, water, triturated with ethanol under heating, dried in vacuo, to give bis spirocompound FPSP183. Yield 51.8%. The structure was confirmed by NMR and MS analysis.

### Example 6

### Compound 357

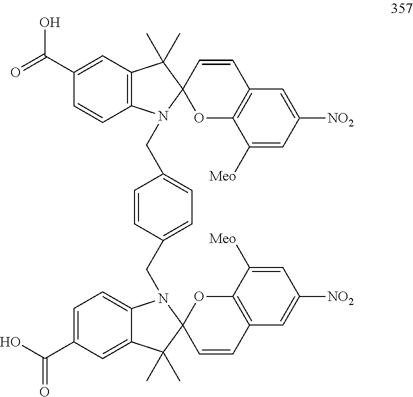

Step 1

Step 1 involves the process described hereinabove as Reaction A.

To a suspension of 4-hydrazinobenzoic acid (25 g, 164 mmol) in ethanol (500 ml) H₂SO₄ (8.8 ml, 16.12 g, 184 mmol) was added portionwise (under cooling with ice-water bath), then methyl isopropyl ketone (14.86 g, 18.46 ml, 173 mmol) was added and the reaction mixture was refluxed for 6 h, cooled to room temperature. After filtration, the solvent was evaporated, a residue was treated with 120 ml of sodium carbonate (sat), then pH was adjusted to 3-4 with acetic acid (glacial) and the mixture was extracted with dichloromethane 4×70 ml. Joined organic phases were dried over Na₂SO₄, passed through short silica column (elution dichloromethane-methanol-2-7%), fractions contained the product were collected, evaporated to dryness to afford solid reddish residue, which was re-crystallized from boiling toluene, washed with hexane, dried in vacuum, giving rise to 26.7 g (80% Yield) of 5-carboxy-2,3,3-trimethyl-indolenine. NMR spectrum conforms to the structure.

Step 2

A mixture of 5-carboxy-2,3,3-trimethyl-indolenine (4.0 g, 19.70 mmol) and α,α′-dibromoxylene (2.0 g, 7.58 mmol) in acetonitrile-toluene (60 ml, 1:2) was refluxed for 90 h. Then a brownish solid was filtered, washed with ether (2×20 ml), triturated with boiling toluene, following by hot filtration, washed with ether, to afford ˜5.8 g of crude material 356 as di-hydrobromide (pink powder). 2.4 g of the product 356 was dissolved in dichloromethane, treated with Na₂CO₃, and then pH of the water layer was adjusted to 3-4 by acetic acid. The organic phase was separated, water layer was extracted twice with dichloromethane, joined organic extracts were dried over Na₂SO₄, evaporated to dryness to afford 1,4-bis((5-carboxy-3,3-dimethyl-2-methyleneindolin-1-yl)methyl)-benzene (quantitative yield), which was subjected to the next step.

Step 3

The process of Step 3 in example 1 was followed except that 1,4-bis((5-carboxy-3,3-dimethyl-2-methyleneindolin-1-yl)methyl)benzene was used instead of 4,4′-bis((3,3-dimethyl-2-methyleneindolin-1-yl)methyl)-1,1′biphenyl. The reaction mixture was refluxed for 2 h in acetonitrile. Crude product was triturated with ethanol overnight, washed with ethanol, dried in vacuo, to afford 1.4 g (51.3%) SP357 as yellow green powder.

### Example 7

### Compound 343

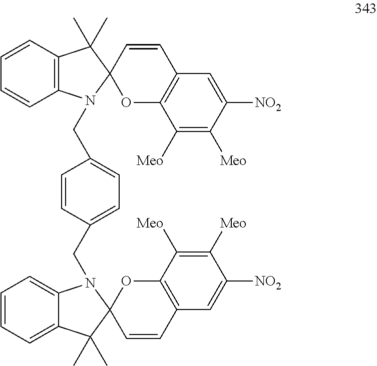

Step 1

Step 1 involves the process described hereinabove as Reaction C. 3,4-dimethoxy-salicylaldehyde (1.5 g, 8.23 mmol) was dissolved in the mixture of acetic acid (5 ml) and dichloromethane (5 ml). The solution was cooled to −10° C. (ice-water NaCl bath).

A solution of fuming nitric acid (0.778 g, 0.512 ml, 1.5 eq) in 2 ml of acetic acid was added slowly by means of dropping funnel at such rate that the temperature was not exceed −5° C. After the reaction was completed (TLC monitoring), the mixture was poured into ice-water (100 ml) under vigorous stirring. The product precipitated was extracted with dichloromethane (3×20 ml), an organic phase was washed with 1M HCl (20 ml), dried over Na₂SO₄, passed through silica pad, evaporated to dryness giving rise to crude yellow product. The product was re-crystallized from ethanol, dried in vacuum. NMR spectrum conforms to the structure of 3,4-dimethoxy-5-nitro-salicylaldehyde.

Step 2.

To a suspension of 1,4-bis((3,3-dimethyl-2-methyleneindolin-1-yl)methyl)-benzene (0.31 g, 0.74 mmol) prepared as described in Example 2 (Step 2) in ethanol (45 ml) 3,4-dimethoxy-5-nitro-salicylaldehyde (0.336 g, 1.479 mmol) was added under stirring. The reaction mixture was refluxed for 2 h, cooled to room temperature, filtered, washed with ethanol, water, ethanol, giving rise to crude product FPSP343 (0.38 g, 51.4%), which was triturated with ethanol, dried in vacuo.

The active crystalline materials of the TTIs were embedded in a suitable matrix including anti-foaming and anti-drying agents.

The abovementioned materials displayed good resistance towards photobleaching. Results for five representative bis-spirocompounds (Scheme B) are presented in Table 1.

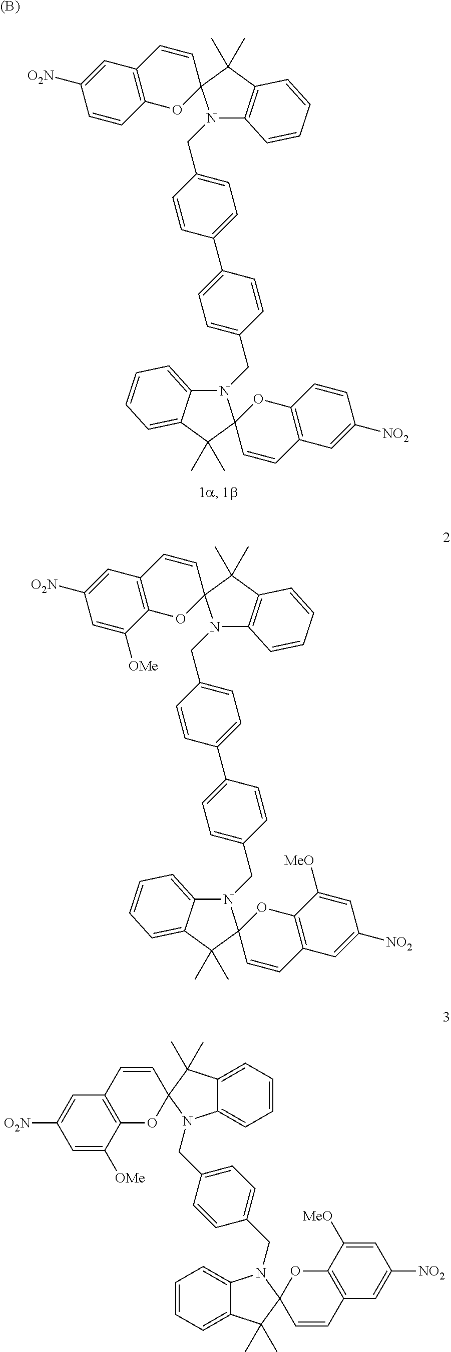

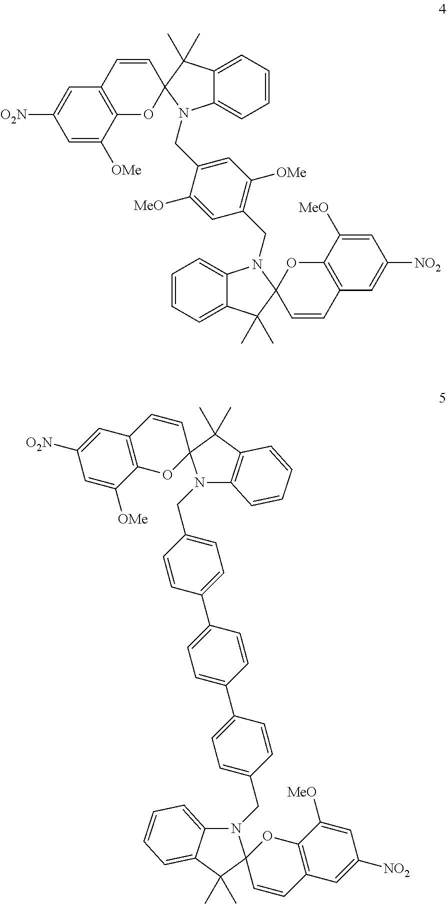

The fading process of photoactivated compounds 1-5 was studied in a time frame of 150 hours. The measurements were performed at T=0° C. The fading process of TTIs, that were exposed to artificial light, followed a linear trend as a function of time (Table 1) with a moderate slope comparatively to the monomeric spiropyran included in the broad application. These results display a clear improvement both in terms of the quality and depth of the activated state's color and in the differentiation of the two coloured states, in comparison to prior art. Moreover, the life time of the activated state is increased and this feature is partially due to the enhanced photostability toward visible light of these compounds (see values for A colour intensity in Table 1).

TABLE 1

| | Fading^{a} | Δ Colour intensity^{c} |
|---|---|---|
| Compound | [Lab/h]^{b} | [Lab]^{b} |
| 1 | 0.0904 | 10 |
| 2 | 0.0634 | 4 |
| 3α | 0.0637 | 6 |
| 3β | 0.0334 | 3 |
| 4 | 0.129 | 10 |
| 5 | 0.0352 | (−2) |

^{a}Colour fading of the samples exposed to artificial light.
^{b}Lab = (L² + a² + b²)^{0.5}.
^{c}decrease in colour intensity after exposure to artificial light for 150 hours.

Stabilization Against Photobleaching

Samples of the pigment were incorporated in identical water based ink, dispersed using a mill under the same conditions. The ink was printed on the same paper substance (LENETTA) and dried in an oven (30° C.) for 24 hrs. The samples were placed on 5 mm glass plates that served as a thermal reservoir and charged using the same light source (lamp 365 nm or LED 365-UV Light Emitting Diode (365 nm)). Two identical samples were prepared and charged from each ink. One system was placed in the dark at 0° C. while the other was exposed to filtered light (cutoff filter 455 nm) of a fluorescent lamp (“OSRAM” DULUX S G23, 900 lm, 11W/840), distance of 30 cm). The samples were measured using a colorimeter (Eye One GretagMacbeth). The CIE Lab values of the charged label that was kept in the dark were compared to the values of an identical label that was exposed to photobleaching light. As is evident from the following graphs, methoxy groups on the nitrophenyl group consistently reduce the photosensitivity of the colored species.

Typically, the spiroaromatic compounds of the invention are incorporated into water based or solvent based ink (in some embodiments) prepared as follows.

Preparation of the Ink Comprising Oligomeric Spiropyrans

Water Based Ink Composition: 10% TTI

Step 1. Polymer Matrix Preparation:
20 g of LS-16 (Ciba® GLASCOL® LS16—an aqueous microemulsion based on a carboxylated acrylic copolymer)
20 g of LS-20 (Ciba® GLASCOL® LS20—an aqueous microemulsion based on a carboxylated acrylic copolymer)
0.25 g of TEGO—TEGO® FOAMEX 845 defoamer emulsion of an organically modified polysiloxane, contains fumed silica)
0.1 g of triethanolamine (TEA)—stir for 1 min

Step 2. Preparation of the Ink Sample
0.2 g of TTI
1.6 g of the Polymer matrix
0.4 g of water (HPLC grade)
The mixture was dispersed on pulverisette (six cycles of 5 min at 600 rpm, twice: six cycles of 5 min at 800 rpm) to give the 10% TTI ink.

Solvent Based Ink Composition: 10% TTI

Step 1. Polyvinyl Butyrate (PVB) Varnish Preparation:
2 g PVB+8 g (10 ml) ethanol
Stir for 2 h to afford a clear solution

Step 2. Solvent Based Ink Concentrate Preparation
0.2 g of TTI
0.5 g of PVB varnish
0.2 g of ethanol
0.1 g of ethyl acetate
Disperse on pulverisette (two cycles of 5 min at 600 rpm) to give an ink concentrate

Step 3. Final Ink Preparation
Add to the ink concentrate:
0.6 g of PVB varnish
0.4 g of ethanol
0.2 g of ethyl acetate
Disperse on pulverisette (six cycles of 5 min at 600 rpm, then twice six cycles of 5 min at 800 rpm) to give the 10% TTI ink

Photobleaching Table at 0° C.
The CIE Lab values of the charged label that was kept in the dark were compared to the
values of an identical label that was exposed to photobleaching light.

| | (L² + a² + b²)^{0.5} | Charging | Time, | (L² + a² + b²)^{0.5} | |
|---|---|---|---|---|---|
| Compound | uncharged | conditions | hrs | filter | Dark |
| 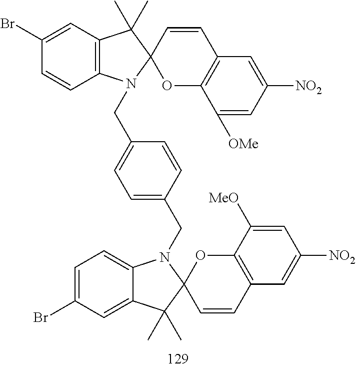 | 89 | 3 min *Tube lamp | 0 20 40 70 100 120 140 160 | 51 55 57 60 62 63 64 65 | 51 52 53 53 54 55 55 55 |
| 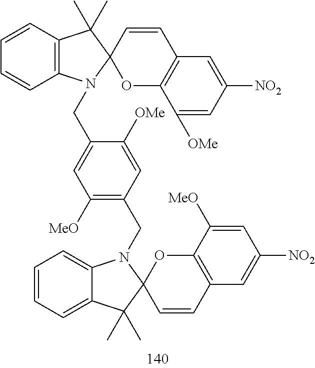 | 95 | 3 min Tube lamp | 0 25 50 75 100 125 150 | 57 60 62 63 65 70 72 | 57 60 60 61 61 62 63 |
| 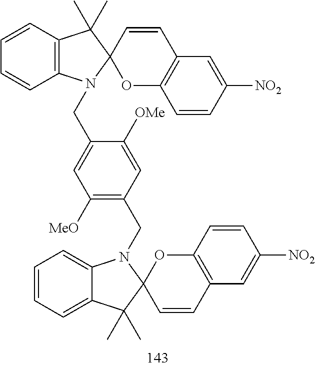 | 87 | 5 min Tube lamp | 0 25 50 75 100 | 57 59 61 63 68 | 57 55 55 56 56 |
| 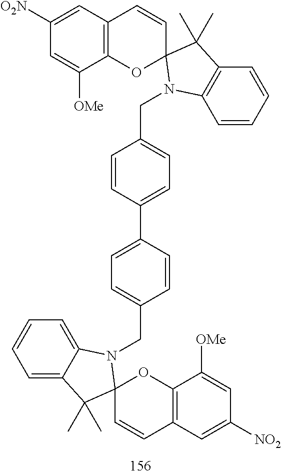 | 84 | 3 min Tube lamp | 0 25 50 75 100 125 150 175 200 225 250 275 | 48 49 49 49 49 50 50 51 52 52 52 53 | 48 49 49 49 49 49 49 49 50 50 50 50 |
| 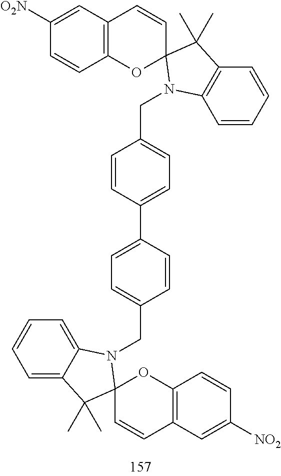 | 86 | 5 min Tube lamp | 0 20 40 70 100 140 | 62 56 58 59 60 65 | 62 55 54 54 55 55 |
| 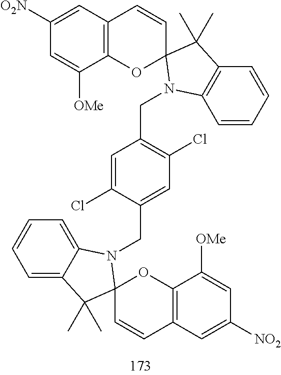 | 93 | 2 min Tube lamp | 0 10 20 50 | 57 62 64 72 | 57 62 63 67 |
| 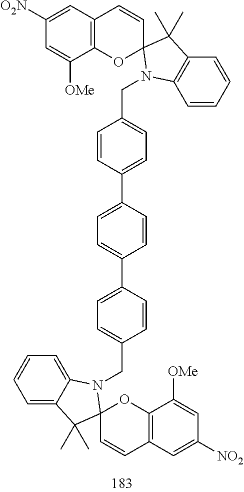 | 87 | 2 min tube lamp | 0 25 50 75 100 150 175 | 45 43 42 43 45 46 46 | 45 46 46 47 48 48 49 |
| 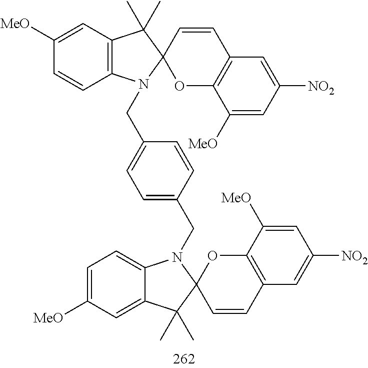 | 65 | 15 sec **LED 365 | 0 20 120 | 53 51 53 | 53 51 52 |
| 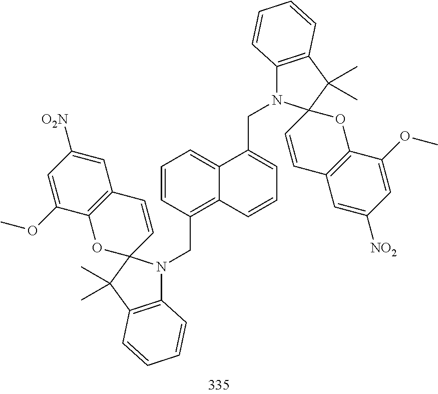 | 65 | 15 sec LED 365 | 0 20 40 60 90 140 160 | 60 65 65 66 67 67 68 | 60 65 67 69 70 72 73 |
| 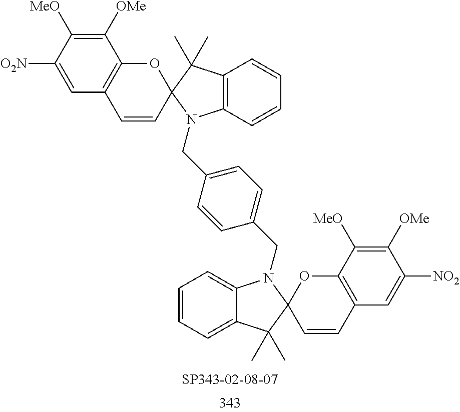 | 88 | 15 sec LED 365 | 0 20 40 60 90 140 160 | 58 73 74 75 76 77 77 | 58 75 80 84 86 87 88 |
| 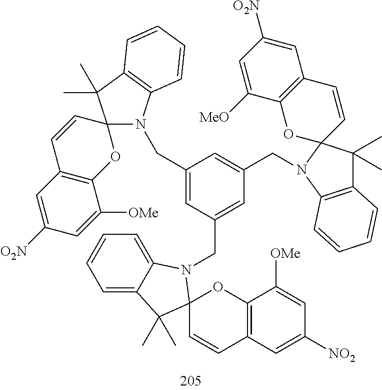 | 76 | 15 sec LED 365 | 0 20 50 70 90 120 170 | 56 57 57 57 57 58 59 | 56 57 57 57 57 58 59 |

*Laboratory UV tube lamp VL—6.LC (6W-365 nm)
**LED 365—UV Light Emitting Diode (365 nm)

Kinetics of the fading processes are presented for two representatives of the oligomeric spiropyrans. The kinetic measurements were performed at various temperatures; the photo-activation of the oligomeric spiropyrans was carried out by irradiating the samples with either a 365 nm LED (about 300 mJ for compound 127) or a tube lamp (about 900 mJ for compound 140). The kinetic data shows that the fading process fits a bi-exponential time-temperature correlation.

Kinetic measurements for the fading process of photo-activated compound 127

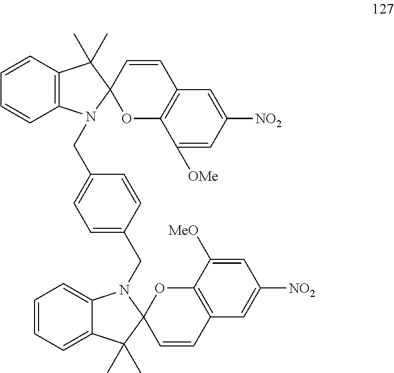

15 sec charging using LED 365 nm

(L² + a² + b²)^{0.5}

| Time hrs | 0° C. | 5° C. | 7° C. | 10° C. | 25° C. |
|---|---|---|---|---|---|
| 0 | 57 | 57 | 57 | 57 | 57 |
| 50 | 60 | 63 | 68 | 69 | 75 |
| 100 | 62 | 67 | 72 | 73 | 77 |
| 150 | 65 | 68 | 73 | 74 | 78 |
| 200 | 67 | 69 | 74 | 75 | 79 |
| 250 | 67 | 70 | 75 | 76 | 80 |
| 300 | 68 | 70 | 75 | 76 | 80 |
| 350 | 68 | 71 | 75 | 76 | 80 |
| 400 | 69 | 71 | 76 | 77 | 80 |

Kinetic measurements for the fading process of photo-activated compound 140

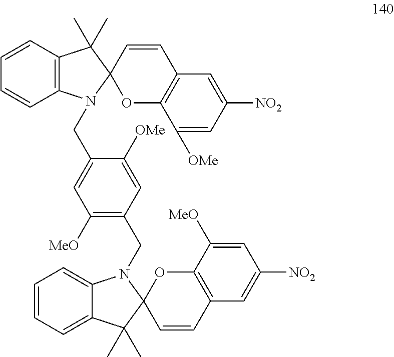

15 sec charging using LED 365 nm

(L² + a² + b²)^{0.5}

| Time hrs | 0° C. | 5° C. | 10° C. | 15° C. | 25° C. |
|---|---|---|---|---|---|
| 0 | 57 | 57 | 57 | 57 | 57 |
| 50 | 60 | 61 | 62 | 85 | 91 |
| 100 | 60 | 63 | 64 | 91 | 91 |
| 150 | 61 | 67 | 68 | 91 | 91 |
| 200 | 61 | 67 | 68 | 92 | |
| 250 | 61 | 68 | 69 | 93 | |

1. A time temperature indicator comprising at least one dimeric or trimeric spiropyran indicator of the formula I or II 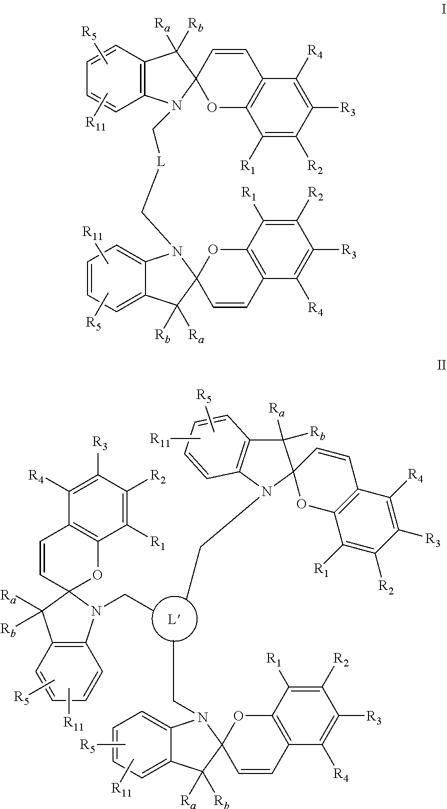
wherein
R₁-R₄ independently of one another is hydrogen, —C₁-C₆ alkoxy, halogen, CF₃, —C₁-C₆ alkyl or —NO₂;
R₅ is hydrogen, halogen, —C₁-C₆ alkoxy, —COOH, —COO—C₁-C₆alkyl, —CF₃ or phenyl;
R₁₁ is hydrogen or R₁₁ and R₅ form together a phenyl ring;
Rₐ is —C₁-C₆ alkyl
R_{b} is —C₁-C₆ alkyl, or together with Rₐ form a 5-6 membered ring
L is a divalent linker;
L′ is a trivalent linker.

2. The time-temperature indicator according to claim 1, comprising at least one trimeric spiropyran indicator of the formula II.

3. The time-temperature indicator according to claim 1, comprising at least one dimeric spiropyran indicator of the formula I.

4. The time-temperature indicator according to claim 3, wherein
R₁ is hydrogen, —C₁-C₆ alkoxy, halogen, —C₁-C₆ alkyl or —NO₂;
R₂ is hydrogen or —C₁-C₆ alkoxy;
R₃ is NO₂ or halogen;
R₄ is hydrogen, —C₁-C₆ alkoxy or halogen;
R₅ is hydrogen, halogen, methoxy or —COOH
R₁₁ is hydrogen,
Rₐ is methyl or ethyl.
R_{b} is methyl or ethyl.
L is a divalent linker.

5. The time-temperature indicator according to claim 1, wherein the at least one dimeric spiropyran indicator compound is selected from the group consisting of the following structural formulae

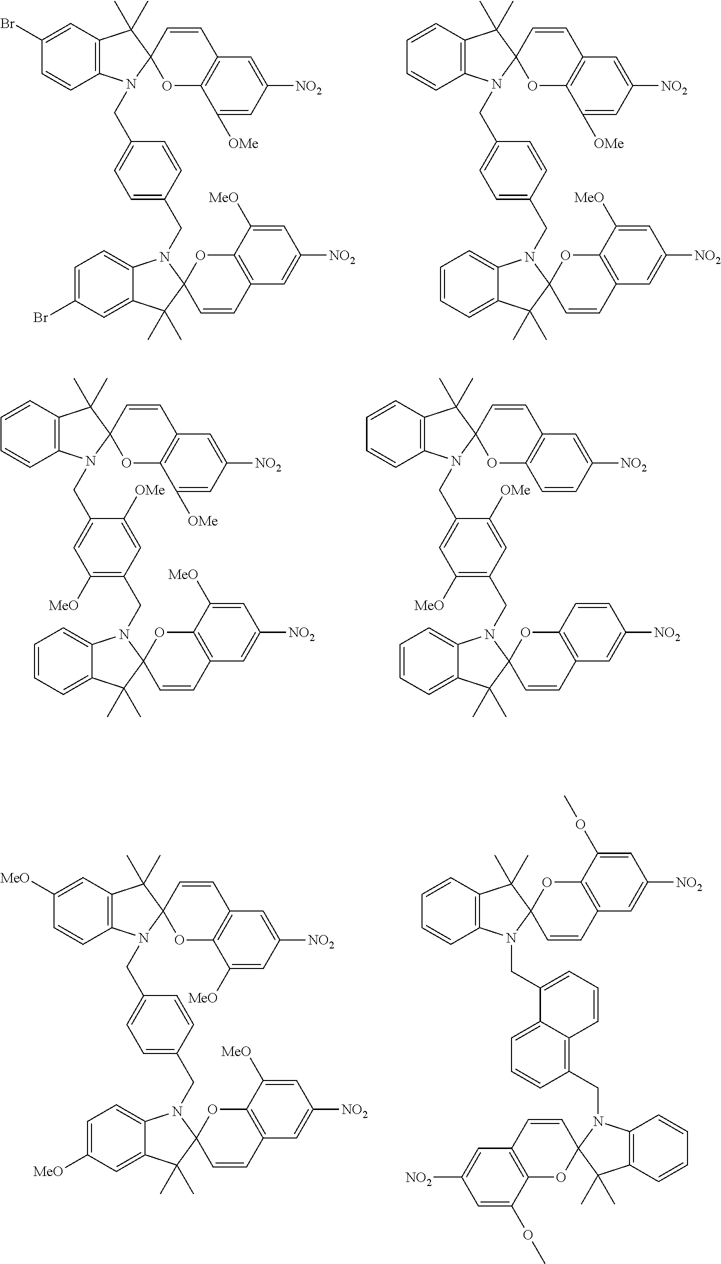

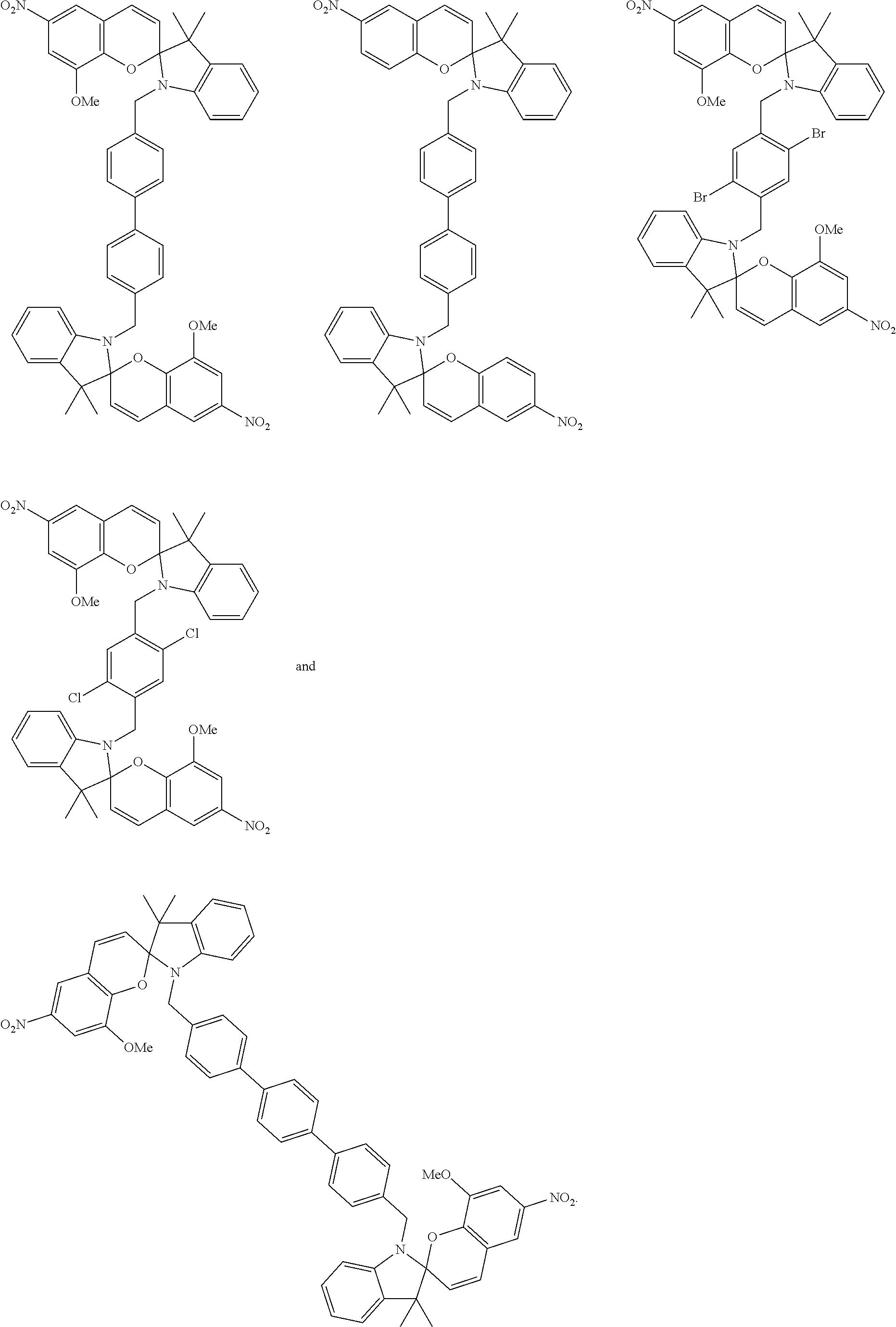

6. A method of manufacturing a time-temperature indicator according to claim 1 comprising at least one of the spiroaromatic indicator compounds of the formula I or II in form of a pigment or a dye; said method comprising the steps of
(a) introducing into a matrix or atop a matrix a dimeric or trimeric spiropyran indicator of the formula I or II and
(b) converting the spiropyran indicator from an original stable state into a metastable state by a process selected from photonic induction, thermal induction, pressure induction, electrical induction, or chemical induction,
(c) optionally applying a protector film.

7. A method of time temperature indication by converting the spiropyran indicator of the formula I or II according to claim 1 from an original stable state into a metastable state by a process selected from photonic induction, thermal induction, pressure induction, electrical induction, or chemical induction and detecting the time temperature dependent re-conversion from the metastable state to the original stable state.

8. The method of claim 7, wherein a color change is detected based on the color difference between said metastable and original state.

9. A printing ink or printing ink concentrate for manufacturing a time temperature indicator comprising at least one spiropyran indicator of the formula (I) or (II) 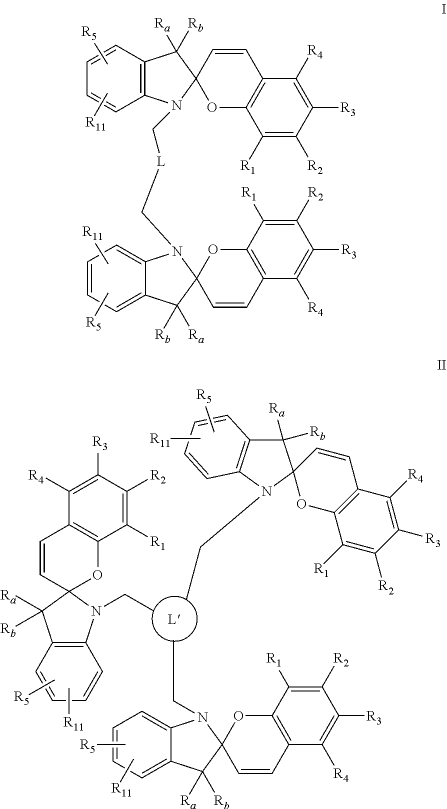
wherein
R₁-R₄ independently of one another is hydrogen, —C₁-C₆ alkoxy, halogen, CF₃, —C₁-C₆ alkyl or —NO₂;
R₅ is hydrogen, halogen, —C₁-C₆ alkoxy, —COOH, —COO—C₁-C₆alkyl, —CF₃ or phenyl;
R₁₁ is hydrogen or R₁₁ and R₅ form together a phenyl ring;
Rₐ is —C₁-C₆ alkyl
R_{b} is —C₁-C₆ alkyl, or together with Rₐ form a 5-6 membered ring
L is a divalent linker;
L′ is a trivalent linker.
